(19) United States
(12) Patent Application Publication
(10) Pub. No.: US 2009/0017016 A1
(43) Pub. Date: Jan. 15, 2009

(54) Treatment and prevention of hypersensitivity and/or anaphylaxis with anti-ige antibodies in patients receiving replacement therapy

(75) Inventors: Sek Chung Fung, Houston, TX (US); Stanley T. Lewis, Pearland, TX (US)

Correspondence Address: Nikolaos C. George;JONES DAY, 222 E. 41st. Street, New York, NY, 10017-6702, US

(73) Assignee: Tanox Inc.

(21) Appl. No.: 11/664,777

(22) PCT Filed: Oct. 4, 2005
(86) PCT No.: PCT/US05/35770

§ 371 Date: Apr. 5, 2007

Related U.S. Application Data
(60) Provisional application No. 60/616,012, filed on Oct. 5, 2004.

(51) Int. Cl. A61K 39/395; A61P 37/08

(52) U.S. Cl. 424/131.1

(57) ABSTRACT

The present invention relates generally to a method of treatment and/or prevention of hypersensitivity including anaphylaxis in patients receiving replacement therapy molecules that elicit an IgE-mediated response by administering an anti-IgE antibody or a binding fragment thereof. Anti-IgE antibodies inhibit IgE-mediated allergic reactions in the mammals and can also lower the risk of an anaphylactic reaction to a replacement therapy molecule. The administration of anti-IgE antibodies over time also downregulates the high-affinity IgE receptor, further decreasing the risk of hypersensitivity and/or anaphylaxis. The anti-IgE antibody binds to circulating or serum IgE and/or membrane form IgE on B-cells, but not to IgE bound to mast cells or basophils, as this may cause crosslinking. This method also reduces the dose and/or frequency of administration of the replacement therapy molecule by reducing or eliminating neutralizing IgE antibodies specific for the replacement therapy molecule.

### BACKGROUND OF THE INVENTION

Drug-induced anaphylaxis has increased in frequency with more widespread use of pharmaceutical agents. Antibacterials are the most usual offenders, penicillins being the most studied. Other compounds commonly causing such reactions include non-steroidal anti-inflammatory drugs, anesthetics, muscle relaxants, latex and radiocontrast media. More recently such reactions have been observed in patients administered replacement therapy molecules, such as, clotting agents including Factor VIII and Factor IX in patients with hemophilia A or B (Shopnick, et al., Transfusion (1996) 36: 358-61; Dioun, et al. B. J. Allergy Clin Immunol (1998) 102: 113-7); human alpha-L-iduronidase in patients with mucopolysaccharidosis (Wraith, et al., J Pediatr (2004) 144: 581-588); human β-glucocerebrosidase in patients with Gaucher's disease (Aviner, et al. Blood Cells Mol Dis (1999) 25: 92-94); and human β-galactosidase A in patients with Fabry disease (Wilcox, et al., Am J Hum Genet (2004) 75: 65-74).

Anaphylaxis is a systemic, severe immediate reaction caused by immunoglobulin (Ig) E-mediated immunological release of mediators from mast cells and basophils.

This immediate hypersensitivity response is based on the production of antibodies of the immunoglobulin class E (IgE antibodies) by B cells which differentiate into antibody secreting plasma cells upon exposure to an agent acting as an allergen, in this case a replacement therapy molecule. The IgE will first sensitize local mast cells; IgE antibodies bind with their constant regions to Fcε receptors on the surface of the mast cells, and then IgE produced from plasma B cells enters the circulation and binds to FCε receptors on both circulating basophils and mast cells in tissues throughout the body. When the bound IgE is subsequently contacted with the allergen, the high affinity IgE receptors (FcεRl) are crosslinked by the binding of allergen causing the cells to degranulate and release a number of anaphylactic mediators such as histamine, prostaglandins, leukotrienes, proteases, etc. It is the release of these substances that is responsible for the clinical symptoms of immediate hypersensitivity, namely contraction of smooth muscle in the respiratory tract, the dilation of small blood vessels and the increase in their permeability to water and plasma proteins, the secretion of mucus, and the stimulation of nerve endings in the skin resulting in itching and pain. In addition, the reaction upon second contact with the allergen is intensified because some B cells form a “memory pool” of surface IgE positive B cells (slgE⁺ B cells) after the first contact with the allergen by expressing IgE on the cell surface. This second exposure can lead to anaphylaxis in some patients.

The treatment of asthma with anti-IgE antibody has been shown to be beneficial for inhibition of allergic reactions by removing the circulating IgE and downregulating the IgE immune response, which is the earliest event in the induction of asthma. As the response of other antibody classes is not affected, both an immediate and a long lasting effect on allergic symptoms are achieved. Early studies of human basophil density showed a correlation between the level of IgE in the plasma of a patient and the number of FcεRl receptors per basophil (Malveaux el al., J. Clin. Invest., 1978, 62:176). They noted that the FcεRl densities in allergic and non-allergic persons range from 10⁴ to 10⁸ receptors per basophil. Later it was shown that treatment of allergic diseases with anti-IgE decreased the amount of circulating IgE to 1% of pretreatment levels (MacGlashan et al., J. Immunol., 1997, 158:1438-1445). MacGlashan analyzed serum obtained from patients treated with whole anti-IgE antibody, which binds free IgE circulating in the serum of the patient. They reported that lowering the level of circulating IgE in a patient resulted in a lower number of receptors present on the surface of basophils. Thus, they hypothesized that FcεRl density on the surface of basophils and mast cells is directly or indirectly regulated by the level of circulating IgE antibody.

This therapeutic approach has been used in treating allergic diseases, such as allergic rhinitis and asthma, using the humanized anti-IgE antibody XOLAIR® (Corne, J. et al., J. Clin. Invest. 1997, 99:879-887; Racine-Poon, A. et al., Clin. Pharmcol. Ther. 1997, 62:675-690; Fahy, J. V. et al., Am. J. Resp. Crit. Care Med. 1997, 155:1824-1834; Boulet, L. P. et al., Am. J. Resp. Crit. Care Med., 1997, 155:1835-1840; Milgrom, E. et al., N. Engl. J. Med., 1999, 341:1966-1973). These clinical data demonstrate that inhibition of IgE binding to its receptors is an effective approach to treating IgE-mediated diseases.

Patients receiving a replacement therapy molecule that exhibit an allergic response to the molecule must undergo numerous infusions in low doses to induce immune tolerance or desensitize the patient. This is very risky in patients exhibiting a severe allergic reaction and can result in anaphylactic shock. Some patients cannot undergo this type of treatment and must rely on alternative, less effective drugs. Patients who have a complete gene deletion resulting in a genetic deficiency are predisposed to this type of anaphylactic risk because they have no tolerance for the drug. These are often life-threatening diseases if drug cannot be administered and yet the treatment is equally life-threatening. Therefore, there is a serious need for a method of treatment to allow administration of the replacement therapy molecule without the risk of hypersensitivity or anaphylaxis.

### SUMMARY OF THE INVENTION

The present invention relates generally to a method of treatment and/or prevention of hypersensitivity and/or anaphylaxis in a mammal administered one or more replacement therapy molecules that elicit an IgE-mediated response by administering an anti-IgE antibody or a binding fragment thereof. The anti-IgE antibodies inhibit IgE-mediated allergic reactions in the mammal thus lowering the risk of an anaphylactic reaction to the replacement therapy molecule. The administration of anti-IgE antibodies over time downregulates the high-affinity IgE receptor (FcεRl), further decreasing the risk of an allergic response.

In the present methods, the anti-IgE antibody binds to circulating or serum IgE and/or IgE bound to B-cells, but preferably does not bind to IgE bound to mast cells or basophils, as this may cause crosslinking. In addition, the anti-IgE antibody also prevents the binding of IgE to the low-affinity IgE receptor (FcεRll) on B cells and antigen presenting cells such as dendritic cells, thereby further reducing the production of allergen-specific IgE by B cells.

The invention also relates to a method for reducing or eliminating an IgE-mediated response in a mammal administered one or more replacement therapy molecules comprising administering an anti-IgE antibody to the mammal.

The present invention also relates to a pharmaceutical composition comprising a replacement therapy molecule and an anti-IgE antibody or a binding fragment, analog, or derivative thereof.

In addition, some IgE antibodies produced by the mammal in response to therapy are neutralizing, thus the efficacy of replacement therapy (RT) is reduced over time. As a result, higher doses of RT molecules are needed to achieve optimal clinical benefits. By administering anti-IgE antibodies that bind to the RT molecule-specific IgE, the amount of RT molecule needed to treat patients is decreased, thus reducing the frequency of drug administration and potentially reducing the patient's cost for therapy.

Diseases to be treated that exhibit an IgE-mediated response include, but are not limited to, genetic deficiency diseases, such as hemophilia A and B, Gaucher's disease, Fabry's disease, mucopolysaccharidosis, diseases involving inborn metabolism errors, and liver enzyme diseases, such as acid-alpha-glucosidase.

Anti-IgE antibodies include, but are not limited to, native, polyclonal, monoclonal, monovalent, bispeciflc, heteroconjugate, multispecific, human, humanized, deimmunized, or chimeric antibodies, single chain antibodies, Fv fragments, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The antibody may be a fragment, such as an Fab, Fab′, F(ab′)₂, Fv, and single binding domain fragments. The antibody may also be a single chain antibody such as a ScFv.

The present invention also involves the use of a composition comprising the anti-IgE antibodies described above in combination with one or more of a pharmaceutically acceptable carrier, diluent, excipient, or stabilizer. The antibodies or compositions useful in the present invention may be administered by one or more of the routes including intravenous, intraperitoneal, inhalation, intramuscular, subcutaneous and oral routes. The antibodies or compositions useful in the present invention may be administered using an inhalation device that delivers to a patient a therapeutically effective amount of an antibody according to the claimed invention.

Other aspects of the invention will become apparent from the detailed description and claims.

### DETAILED DESCRIPTION OF THE INVENTION

Terms used throughout this application are to be construed with ordinary and typical meaning to those of ordinary skill in the art. However, the following terms are to be given the broadest reasonable construction as indicated by the descriptions provided below.

The term “antibody” as used herein is used in its broadest sense and includes an immunoglobulin molecule or an immunologically active portion of an immunoglobulin molecule, i.e., a molecule that contains an antigen binding site that immunospecifically binds an antigen. The immunoglobulin molecule can be of any type (e.g., IgG, IgE, IgM, IgD, IgA or IgY), class (e.g., IgG1, IgG2, IgG3, IgG4, IgA1 or IgA2) or subclass of immunoglobulin molecule. Antibodies include, but are not limited to, native, polyclonal, monoclonal, monovalent, bispecific, heteroconjugate, multispecific, human, humanized, deimmunized, or chimeric antibodies. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. However, “antibody” as used herein includes single-domain antibodies having only one chain, either a heavy or a light chain with specificity for the target, as well as single chain antibodies, Fv fragments, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above.

As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The term “antibody fragment” as used herein includes a portion of a full-length antibody, generally the target binding or variable region. Examples of antibody fragments include Fab, Fab′, F(ab′)₂ and Fv fragments. The term “binding fragment” of an antibody means a compound having qualitative biological activity or detectable immunological activity in common with a full-length antibody. For example, a binding fragment of an anti-IgE antibody is one that binds to an IgE immunoglobulin and/or prevents or substantially reduces the ability of such molecule to bind to the high affinity receptor FcεRl and or the low affinity IgE receptor FcεRll.

The term “monoclonal antibody” as used herein includes an antibody obtained from a population of substantially homogeneous antibodies. The individual antibodies comprising the population are identical except for possible naturally occurring mutations that may be present in minor amounts. Monoclonal antibodies are highly specific, being directed against a single targeted site. Furthermore, in contrast to conventional (polyclonal) antibody preparations which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use in the present invention may be isolated from phage antibody libraries using well known techniques. The parent monoclonal antibodies to be used in accordance with the present invention may be made by the hybridoma method first described by Kohler and Milstein, Nature 256, 495 (1975), or may be made by recombinant methods.

“Humanized” forms of non-human (e.g. murine) antibodies include chimeric immunoglobulins, immunoglobulin chains or fragments thereof (such as Fv, Fab, Fab′, F(ab′)₂ or other target-binding subsequences of antibodies) that contain minimal sequence derived from non-human immunoglobulin. In general, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin template chosen.

The term “anti-IgE antibody” as used herein includes an antibody that binds to one or more IgE molecules and inhibits or substantially reduces the binding of the IgE molecules to the high affinity receptor FcεRl and or the low affinity receptor FcεRll. An anti-IgE antibody may bind to any portion of an IgE molecule, such as to the constant region or the variable region of an IgE molecule. An anti-IgE antibody may be able to bind to a variety of species of IgE molecules or may bind to a particular IgE species, such as by binding to the binding site of an IgE antibody.

The term “IgE-mediated” as used herein includes a condition or disease that is characterized by the overproduction of the immunoglobulin IgE and/or immunological and inflammatory reactions which are IgE-dependent. It includes but is not limited to conditions associated with anaphylactic hypersensitivity.

A RAST test may be done to determine whether a mammal is in need of such treatment. A RAST test (short for radioallergosorbent test) is a blood test used to determine whether a patient is allergic to a given compound. A RAST test detects the amount of IgE in a patient's blood that reacts specifically with a given allergen. The present invention contemplates that a RAST test may be done using a replacement therapy molecule as the allergen.

### Target Preparation

Soluble targets or fragments thereof can be used as immunogens for generating antibodies, in this case IgE or a fragment thereof. The target may be produced recombinantly or made using synthetic methods. The target may also be isolated from a natural source.

### Anti-IgE Antibody Generation

The antibodies useful in the present invention may be generated by any suitable method known in the art. The antibodies used in the present invention may comprise monoclonal antibodies. Methods of preparing antibodies are known to the skilled artisan (Harlow, et al., Antibodies: a Laboratory Manual, (Cold spring Harbor Laboratory Press, 2nd ed. (1988), which is hereby incorporated herein by reference in its entirety).

For example, an immunogen as described above may be administered to various host animals including, but not limited to, rabbits, mice, rats, etc., to induce the production of sera containing polyclonal antibodies specific for the antigen. The administration of the immunogen may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Additional examples of adjuvants which may be employed Includes the MPL-TDM adjuvant (monophosphoryl lipid A, synthetic trehalose dicorynomycolate). The immunization protocol may be selected by one skilled in the art without undue experimentation. Such adjuvants are also well known in the art.

Typically, the immunogen and/or adjuvant will be injected in the mammal by multiple subcutaneous or intraperitoneal injections, though they may also be given intramuscularly, and/or intravenously. The immunogen may include a human IgE polypeptide or a fusion protein or variants thereof. Depending upon the nature of the polypeptides (i.e., percent hydrophobicity, percent hydrophilicity, stability, net charge, isoelectric point etc.), it may be useful to conjugate the immunogen to a protein known to be immunogenic in the mammal being immunized. Such conjugation includes either chemical conjugation by derivatizing active chemical functional groups to both the antibodies useful in the present invention and the immunogenic protein such that a covalent bond is formed, or through fusion-protein based methodology, or other methods known to the skilled artisan. Examples of such immunogenic proteins include, but are not limited to keyhole limpet hemocyanin, ovalbumin, serum albumin, bovine thyroglobulin, soybean trypsin inhibitor, and promiscuous T helper peptides. Various adjuvants may be used to increase the immunological response as described above.

Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2.sup.nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies include, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo.

In a hybridoma method, a mouse, a humanized mouse, a mouse with a human immune system, hamster, or other appropriate host animal, is typically immunized with an immunogen to elicit lymphocytes that produce or are capable of producing antibodies that will specifically bind to IgE. Alternatively, lymphocytes may be immunized in vitro with the antigen.

Generally, in making antibody-producing hybridomas, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. For example, immortalized cell lines include murine myeloma lines, which can be obtained, for instance, from the Salk Institute Cell Distribution Center, San Diego, Calif. and the American Type Culture Collection, Manassas, Va. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against an IgE. The binding specificity of monoclonal antibodies produced by the hybridoma cells can be determined by immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody to IgE can, for example, be determined by the Scatchard analysis (Munson et al., Anal. Biochem, 107:220 (1980)).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640.

The monoclonal antibodies secreted by the subclones may be isolated or purified from the culture medium by conventional immunoglobulin purification procedures such as, for example, protein A-sepharose, hydroxyapatite chromatography, gel exclusion chromatography, gel electrophoresis, dialysis, or affinity chromatography.

A variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as NSO cells, Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a bivalent antibody capable of binding to more than one type of antigen.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively, the relevant cysteine residues are substituted with another amino acid residue or are deleted so as to prevent cross-linking.

Antibody fragments that recognize specific epitopes may be generated by known techniques. For example, Fab and F(ab′)₂ fragments useful in the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For some uses, including in vivo use of antibodies in humans and in vitro detection assays, it may be preferable to use chimeric, humanized, or human antibodies, A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more complementarity determining regions (CDRs) from the non-human species and framework (FR) regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties).

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also comprises at least a portion of an immunoglobulin constant region (Fc), typically that of a human immunoglobulin (Jones et al., Nature, 321:522-525 (1986); Riechmann et al., Nature 332:323-329 (1988)1 and Presta, Curr. Op. Struct. Biol., 2:593-596 (1992).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of Cole et al., and Boerder et al., are also available for the preparation of human monoclonal antibodies (Cole et al., Monoclonal Antibodies and Cancer Therapy, Alan R. Riss, (1985); and Boemer et al., J. Immunol., 147(1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies.

The transgenic mice are immunized in the normal fashion with a selected antigen. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Also human monoclonal antibodies could be made by immunizing mice transplanted with human peripheral blood leukocytes, splenocytes or bone marrows (e.g. Trioma techniques of XTL). Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Heteroconjugate antibodies are also contemplated as useful in the present invention. Heteroconjugate antibodies are composed of two covalently joined antibodies. Such antibodies have, for example, been proposed to target immune system cells to unwanted cells (U.S. Pat. No. 4,676,980). It is contemplated that the antibodies may be prepared in vitro using known methods in synthetic protein chemistry, including those involving cross-linking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Identification of Anti-IgE Antibodies

Candidate anti-IgE antibodies were tested and can be identified by enzyme linked immunosorbent assay (ELISA), Western immunoblotting, or other immunochemical techniques. Assays performed to characterize the individual antibodies included: (1) Inhibition of IgE binding to FcεRl bearing mast cells and basophils, (2) Inhibition of IgE binding to FcεRll bearing dendritic cells and B cells, (3) Binding to membrane IgE-bearing B cells, and (4) Inhibition of IgE binding to recombinant soluble FcεRl protein on solid-phase. One or more assays can be used to select an individual antibody as an anti-IgE antibody.

Antibodies useful in the invention include, but are not limited to, native, polyclonal, monoclonal, monovalent, bispecific, heteroconjugate, multispecific, human, humanized, deimmunized, or chimeric antibodies, single chain antibodies, Fv fragments, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above.

The antibodies may be human antigen-binding antibody fragments and include, but are not limited to, Fab, Fab′ and F(ab′)₂, Fd, single-chain Fvs (scFv), single-chain antibodies, disulfide-linked Fvs (sdFv) and fragments (which are examples of binding fragments) comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments comprising any combination of variable region(s) with a hinge region, CH1, CH2, and CH3 domains. The antibodies of the invention may be from any animal origin including birds and mammals. Preferably, the antibodies are human, non-human primates, rodents (e.g., mouse and rat), donkey, sheep, rabbit, goat, guinea pig, camel, horse, or chicken.

Antibodies used in the present invention may be described or specified in terms of the epitope(s) or portion(s) of IgE which they recognize or specifically bind. The epitope(s) or polypeptide portion(s) may be specified as described herein, e.g., by N-terminal and C-terminal positions, by size in contiguous amino acid residues, or those published in WO05/075504, which are incorporated here by reference.

### Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24: 107-117 (1992) and Brennan et al., Science 229: 81 (1985)). However, these fragments can now be produced directly by recombinant host cells. For example, the antibody fragments can be isolated from an antibody phage library. Alternatively, F(ab′)₂-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). (PCT patent application WO 93/16185). Binding fragments with respect to antibodies include Fv, F(ab) and F(ab′)₂ fragments. An “Fv” fragment contains a complete target recognition and binding site. It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. Single-chain Fv (ScFv) antibody fragments comprise the V_{H} and V_{L} domains of an antibody, wherein these domains are present in a single polypeptide chain. Generally, the Fv polypeptide further comprises a polypeptide linker between the V_{H} and V_{L} domains that enables the ScFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

### Methods of Producing Anti-IgE Antibodies

The antibodies useful in the invention can be produced by any method known in the art for the synthesis of antibodies, in particular, by chemical synthesis or preferably, by recombinant expression techniques.

Recombinant expression of anti-IgE antibodies, or fragments thereof, (e.g., a heavy or light chain of an antibody or a single chain antibody), requires construction of an expression vector containing a polynucleotide that encodes the antibody or a fragment of the antibody. Once a polynucleotide encoding an antibody molecule or a heavy or light chain of an antibody, or portion thereof, of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Thus, replicable vectors comprising a nucleotide sequence encoding an antibody molecule useful the invention, or a heavy or light chain thereof, or a heavy or light chain variable domain, operably linked to a promoter may be used to prepare anti-IgE antibodies. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. In preferred embodiments for the expression of double-chained antibodies, vectors encoding both the heavy and light chains may be co-expressed in the host cell for expression of the entire immunoglobulin molecule.

A variety of host-expression vector systems may be utilized to express the antibody molecules useful in the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., Baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as E. coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors [\which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest is ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, COS, 293, 3T3, or myeloma cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl . Acad. Sci. USA 48:202 (1992)), and adenine phosphoribosyltransferase (Lowy et al., Cell 22:817 (1980)) genes can be employed in tk, hgprt or aprt-cells, respectively. Also, antimetabolite resistance can be used as the basis of selection for the following genes: dhfr, which confers resistance to methotrexate (Wigler et al., Proc. Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoll et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, “The use of vectors based on gene amplification for the expression of cloned genes in mammalian cells” (DNA Cloning, Vol. 3. Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an anti-IgE antibody has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and size-exclusion chromatography), centrifugation, differential solubility, or by any other standard technique for the purification of proteins. In addition, the antibodies or fragments thereof can be fused to heterologous polypeptide sequences described herein or otherwise known in the art, to facilitate purification.

The present invention encompasses the use of anti-IgE antibodies or fragments thereof recombinantly fused or chemically conjugated (including both covalently and non-covalently conjugations) to a polypeptide. Fused or conjugated antibodies useful in the present invention may be used in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., Proc. Natl. Acad. Sci. 89:1428-1432 (1992); Fell et al., J. Immunol. 146:2446-2452(1991), which are incorporated by reference in their entireties.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

### Pharmaceutical Formulations

Therapeutic formulations of anti-IgE antibodies or fragments thereof may be prepared for storage as lyophilized formulations or aqueous solutions by mixing the antibody having the desired degree of purity with optional “pharmaceutically-acceptable” carriers, excipients or stabilizers typically employed in the art (all of which are termed “excipients”). For example, buffering agents, stabilizing agents, preservatives, isotonifiers, non-ionic detergents, antioxidants and other miscellaneous additives. (See Remington's Pharmaceutical Sciences, 16th edition, A. Osol, Ed. (1980)). Such additives must be nontoxic to the recipients at the dosages and concentrations employed. Therapeutic formulations may also include a combination of the anti-IgE antibody or fragment and the replacement molecule(s).

Buffering agents help maintain the pH in the range which approximates physiological conditions. They are preferably present at concentration ranging from about 2 mM to about 50 mM. Suitable buffering agents for use with the antibodies useful in the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be mentioned phosphate buffers, histidine buffers and trimethylamine salts such as Tris.

Preservatives may be added to retard microbial growth, and may be added in amounts ranging from 0.2%-1% (w/v). Suitable preservatives for use with the antibodies useful in present invention include phenol, benzyl alcohol, meta-cresol, methyl paraben, propyl paraben, octadecyldimethylbenzyl ammonium chloride, benzalconium halides (e.g., chloride, bromide, iodide), hexamethonium chloride, alkyl parabens such as methyl or propyl paraben, catechol, resorcinol, cyclohexanol, and 3-pentanol.

Isotonicifiers sometimes known as “stabilizers” may be added to ensure isotonicity of liquid compositions and include polhydric sugar alcohols, preferably trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran. Stabilizers may be present in the range from 0.1 to 10,000 weights per part of weight active protein.

Non-ionic surfactants or detergents (also known as “wetting agents”) may be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic® polyols, polyoxyethylene sorbitan monoethers (TWEEN®-20, TWEEN®-80, etc.). Non-ionic surfactants may be present in a range of about 0.05 mg/ml to about 1.0 mg/ml, preferably about 0.07 mg/ml to about 0.2 mg/ml.

Additional miscellaneous excipients include bulking agents, (e.g. starch), chelating agents (e.g. EDTA), antioxidants (e.g., ascorbic acid, methionine, vitamin E), and cosolvents. The formulation herein may also contain more than one active compound as necessary for the particular indication being treated, preferably those with complementary activities that do not adversely affect each other. For example, it may be desireable to further provide an immunosuppressive agent. Such molecules are suitably present in combination in amounts that are effective for the purpose intended. The active ingredients may also be entrapped in microcapsule prepared, for example, by coascervation techniques or by interfacial polymerization, for example, hydroxymethylcellulose or gelatin-microcapsule and poly-(methylmethacylate) microcapsule, respectively, in colloidal drug delivery systems (for example, liposomes, albumin micropheres, microemulsions, nano-particles and nanocapsules) or in macroemulsions. Such techniques are disclosed in Remington's Pharmaceutical Sciences, 16th edition, A. Osal, Ed. (1980).

The formulations to be used for in vivo administration are sterile. This is readily accomplished, for example, by filtration through sterile filtration membranes. Sustained-release preparations may be prepared. Suitable examples of sustained-release preparations include semi-permeable matrices of solid hydrophobic polymers containing the antibody variant, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C. resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl, residues, lyophilizing from acidic solutions, controlling moisture content, using appropriate additives, and developing specific polymer matrix compositions.

The amount of antibody or fragment thereof that will be effective in the treatment of a particular disorder or condition will depend on the nature of the disorder or condition, and can be determined by standard clinical techniques. Where possible, it is desireable to determine the dose-response curve and the pharmaceutical compositions of the invention first in vitro, and then in useful animal model systems prior to testing in humans.

In a preferred embodiment, an aqueous solution of antibody or fragment thereof is administered by subcutaneous injection. Each dose may range from about 0.5 mg to about 50 mg per kilogram of body weight, or more preferably, from about 3 mg to about 30 mg per kilogram body weight.

The dosing schedule for subcutaneous administration may vary from once a month to daily depending on a number of clinical factors, including the type of disease, severity of disease, and the subject's sensitivity to the therapeutic agent.

### Therapeutic Applications of the Invention

An anti-IgE antibody, with or without a therapeutic moiety conjugated to it, administered alone or in combination with cytotoxic factor(s) or in combination with the replacement therapy molecule(s) is used as a therapeutic. The present invention includes antibody-based therapies that involve administering anti-IgE antibodies to an animal, preferably a mammal, and most preferably a human, for treating or preventing IgE-mediated diseases, disorders, or conditions, associated with replacement therapy.

Anti-IgE antibodies useful in the present invention may be used therapeutically in a variety of diseases. The antibodies may be used in the treatment of any disease in which a replacement therapy molecule is administered to a patient that elicits an IgE-mediated response thereby reducing the risk of anaphylactic shock or relieving hypersensitivity symptoms associated with the release of allergic mediators. These antibodies may also reduce the frequency of treatment and/or dose of the replacement therapy molecule by eliminating neutralizing IgE molecules specific for the molecule administered. It is preferred to use high affinity and/or potent in vivo inhibiting and/or neutralizing antibodies against IgE, fragments or regions thereof, for therapy of disorders, and preferably antibodies applicable for therapy in the present method should not react with IgE bound on sensitized mast cells and basophils, but should retain the capability to recognize soluble IgE and membrane IgE on B cells.

Therapeutic compounds useful in the invention include, but are not limited to, anti-IgE antibodies (including fragments, analogs and derivatives thereof as described herein) and nucleic acids encoding antibodies. Antibodies useful in the invention may be provided in pharmaceutically acceptable compositions as known in the art or as described herein. Such IgE isotype-specific antibodies have been described e.g. by Chang et al. (Biotechnology 8, 122-126 (1990)), in European Patent No. EP0407392, and several U.S. Patents, including U.S. Pat. Nos. 5,449,760, 5,422,258, and 5,614,611. Other antibodies useful in the present invention include high affinity anti-IgE antibodies as described in WO04070010 and WO04070011, as well as the anti-IgE antibodies described in U.S. Pat. Nos. 5,965,709; 5,994,511, 6,172,213; 6329,509; 6,682,735; and 6,761,889. All of these patents and applications are incorporated herein by reference.

The anti-IgE antibodies may be administered alone or in combination with other types of treatments, such as immunotherapy, bronchodilators, anti-histamines, or anti-leukotrienes.

The anti-IgE antibodies may be administered before, during or after replacement therapy. As mentioned above, replacement therapy is used for treating patients with hemophilia A or B, mucopolysaccharidosis, Gaucher's disease, and Fabry disease, as well as Pompe disease and other genetic or acquired deficiencies of biological proteins. Replacement therapy can be given on an acute or long-term basis. Replacement therapy includes, but are not limited to, enzyme replacement therapy, IgG replacement therapy, and hormone replacement therapy. A common example of replacement therapy is the injection of low or missing clotting factors directly into the bloodstream to treat hemophilia.

Anti-IgE antibodies may be administered before, during or after the administration of one or more replacement therapy molecules. Replacement therapy molecules include, but are not limited to, clotting agents including Factor VIII and Factor IX; human alpha-L-iduronidase; laronidase; human β-glucocerebrosidase; imiglucerase; alglucerase; human β-galactosidase A; agalsidase beta; human alpha-glucosidase; alglucosidase alfa; or any protein administered to replace a deficient protein in a mammal and which may cause an IgE-mediated response. Replacement therapy molecules can include molecules delivered for or through gene therapy. For example, human β-glucocerebrosidase can be delivered as a replacement therapy molecule by use of gene therapy.

The invention provides methods of treatment and prevention of hypersensitivity and/or anaphylaxis by administration to a mammal of an effective amount of an anti-IgE antibody or pharmaceutical composition comprising an anti-IgE antibody. In a preferred aspect, the antibody is substantially purified (e.g., substantially free from substances that limit its effect or produce undesired side-effects).

Various delivery systems are known and can be used to administer an antibody useful in the present invention, including e.g., injection, encapsulation in liposomes, microparticles, microcapsules, recombinant cells capable of expressing the compound, receptor-mediated endocytosis (see, e.g., Wu et al., J. Biol. Chem. 262:4429-4432 (1987)), construction of a nucleic acid as part of a retroviral or other vector, etc.

The anti-IgE antibody can be administered to the mammal in any acceptable manner. Methods of introduction include but are not limited to intradermal, intramuscular, intraperitoneal, intravenous, subcutaneous, intranasal, epidural, inhalation and oral routes. The antibodies or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the therapeutic antibodies or compositions into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. The antibody may also be administered into the lungs of a patient in the form of a dry powder composition (See e.g., U.S. Pat. No. 6,514,496).

In a specific embodiment, it may be desirable to administer the therapeutic antibodies or compositions useful in the invention locally to the area in need of treatment; this may be achieved by, for example, and not by way of limitation, local infusion, topical application, by injection, by means of a catheter, by means of a suppository, or by means of an implant, said implant being of a porous, non-porous, or gelatinous material, including membranes, such as sialastic membranes, or fibers. Preferably, when administering an antibody, care must be taken to use materials to which the antibody does not absorb.

In another embodiment, the antibody can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the antibody can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target.

The amount of the antibody that will be effective in the treatment or prevention of an IgE-mediated response in mammals receiving replacement therapy molecules can be determined, for example, in standard clinical studies. The antibody can be administered in treatment regimes consistent with the disease, e.g., a single or a few doses over one to several days to ameliorate a disease state or periodic doses over an extended time to prevent hypersensitivity or threat of anaphylaxis. In addition, in vitro assays may optionally be employed to help identify optimal dosage ranges. The precise dose to be employed in the formulation will also depend on the route of administration, and the seriousness of the disease or disorder, and can be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patients body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and/or less frequent administration is often possible. Further, the dosage and frequency of administration of anti-IgE antibodies may be reduced by enhancing uptake and tissue penetration (e.g., into the brain) of the antibodies by modifications such as, for example, lipidation.

In addition, the antibodies useful in the present invention may be conjugated to various effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387. An antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Since conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

### EXAMPLES

The following examples are offered by way of illustration and not by way of limitation.

### Example 1

### Production and Screening of TES-C21 and TESC-2

Male Balb/c mice were immunized several times with polyclonal human IgE purified from sera (provided by Ventrex). The IgE was combined with a suitable adjuvant. Mice were sacrificed after the last injection of immunogen and the spleens were removed for preparing single cell suspensions for fusion with myeloma cells. The spleen cells were fused with Sp2/0 cells using a fusion mixture of polyethylene glycol 1450 (Kodak), CMF-PBS and DMSO. DMEM was added after the cell suspensions were combined.

The hybridomas resulting from the fusion were then screened by enzyme-linked immunosorbent assay (ELISA) against human IgE bound to an Immulon 2 plate. One of these hybridomas produced the murine anti-IgE antibody TES-C21.

Sp 2/0 cells were co-transfected with nucleic acid sequences encoding the variable regions of TES-C21 H and L-chains, and human γ1 and κ constant regions, and aliquoted into 96 well plates for selection. Supernatants were screened for secretion of human IgG which bound to human IgE.

The transfectoma cells were adapted to growth in serum-free medium. The resulting chimeric clone TESC-2 was then purified from medium of confluent cultures using an immobilized protein A column.

TES-C21 was further screened by ELISA and confirmed to be specific for human IgE, and did not cross-react with IgG, IgM, IgA, IgD, human serum albumin, transferrin or insulin. TES-C21 bound equally well to various human IgE molecules. TES-C21 bound to the IgE-secreting cell lines SKO-007, U266 and SE44 in a dose-dependent manner, indicating binding to human membrane IgE. But TES-C21 did not bind to human B cell lines bearing surface IgM, IgD, IgG, or IgA, or to a T cell line, or to the parent murine cell line of SE44, or to a murine cell line secreting chimeric human IgG. TES-C21 also did not bind to IgE on low affinity FcεRll receptors present on a wide variety of cell types. It also did not induce histamine release from freshly prepared human blood basophils, on which the FcεRl are armed with IgE. At 10 μg/ml, TES-C21 is able to inhibit completely the binding of 1 μg of IgE to FcεRll.

### Example II

### Determination of Binding Selectivity

TESC-2 and TES-C21 bind equally well to IgE bound to microtiter plates. This was demonstrated as follows. Immulon 2 plates were coated with HIV-1 gp120 derived R15 K peptide-ovalbumin conjugate and SE44 (a chimeric human IgE bearing the variable regions of an antibody which binds the HIV-1 gp120 derived peptide R15K) was bound to the immobilized antigen. TES-C21 or TESC-2 at various concentrations was added. Binding was detected using either horseradish peroxidase (“HRP”)-conjugated goat antimouse IgG (for TES-C21) or HRP-goat antihuman IgG, Fc (for TESC-2). It was determined that TESC-2 and TES-C21 have the same relative affinity for IgE bound to microtiter plates.

TESC-2 and TES-C21 also were shown to bind equally to IgE-producing cells (SKO-007). This was demonstrated by incubating such cells at 2×10⁶ cells/100 μl PBS-1% goat serum at various antibody concentrations at 0° C. for 30 minutes. Binding of TES-C21 was detected using FITC-goat (Fab)₂ antimouse IgG; binding of TESC-2 was detected using FITC-goat (Fab)₂ antihuman IgG. Binding was quantitated by fluorescence flow cytometry using a Coulter Epics V. The FITC intensity gate was set to yield 10% +0.5% positive cells in the absence of primary immunoglobulins.

It was found that neither TES-C21 nor TESC-2 bound to IgE which was bound to low affinity IgE receptors. The possibility that TESC-2 recognized IgE complexed with CD23 was studied using cells of an IgG-secreting human lymphoblastoid line, IM-9. The presence of CD23 on IM-9 cells was confirmed by their strong staining with anti-Leu 20, a MAb specific for CD23. IM-9 cells were incubated with 5 to 10 μg/ml of human IgE, washed, and then incubated with biotin-labeled TESC-2 or a positive control anti-IgE MAb TES-19, followed by FITC-streptavidin and analyzed by flow cytometry.

Both chimeric TESC-2 and murine TES-C21 were shown to inhibit binding of IgE to FcεRll. The antibodies were preincubated at various concentrations with 20 μg IgE-SE44 for 1 hour at 37° before addition of IM-9 cells bearing FcεRll. Binding to IgE was detected using biotinylated TES-19 and FITC-streptavidin and quantitated by fluorescence flow cytometry.

To negate the possibility that immune complexes of TESC-2 and IgE were formed during their preincubation in these experiments, thereby yielding false positives, it was confirmed that these immune complexes also did not bind to FcεRll, using biotin-labeled TESC-2 or FITC goat anti-human IgE (with TES-C21).

### Example III

### Histamine Release Assays

As shown in Table 1 below, neither TESC-2 nor TES-C21 induces histamine release from freshly prepared human blood basophils, on which the FcεRl are armed with IgE. Due to the variable release of mediators from basophils of different donors, the antibodies were examined at multiple concentrations on basophil preparations from multiple donors. No induction of histamine release by TESC-2 or TES-C21 was observed. However, cross-linking of surface IgE with polyclonal goat anti-IgE antibodies induced histamine release.

TABLE 1
Net Histamine Release

| Antibody | Conc. (μg/ml) | Donor 1 | Donor 2 | Donor 3 | Donor 4 |
|---|---|---|---|---|---|
| Polyclonal goat | 0.1 | 70 | 64 | 55 | 81 |
| anti-human IgE | | | | | |
| TESC-2 | 0.4 | 0 | 2 | | |
| | 2 | 0 | 3 | | |
| | 10 | 0 | 2 | | |
| | 50 | 0 | 2 | | |
| TES-C21 | 0.4 | | | 1 | 0 |
| | 2 | | | 1 | 0 |
| | 10 | | | 0 | 0 |
| | 50 | | | 1 | 0 |

To address the possibility that TES-C21 might bind to basophils and induce cross-linking of the receptors to induce histamine release, upon introduction of a cross-linking antigen or other agent, a secondary antibody was used for crosslinking. Since anti-human IgG alone can induce histamine release due to the fact that there are human IgG already bound to the FcγR on the isolated basophils, only the murine antibody TES-C21 was used in these experiments. The crosslinking goat antimouse IgG enhances histamine release induced by suboptimal concentrations of a positive control mouse anti-human IgE which does not inhibit the binding of human IgE to FcεRl. However, TES-C21 did not induce histamine under these very permissive conditions.

A humanized version of the murine antibody TES-C21 was made, as described in detail in Australian Patent No. 675449, granted May 25, 1997. Similar procedures can be followed to produce other humanized anti-IgE antibodies. Several transfectomas producing humanized anti-IgE antibodies suitable for use with the invention are available from the ATCC under the following accession numbers: 11130; 11131; 11132; 11133. An anti-IgE antibody similar to that produced from the transfectoma deposited under accession number 11131 is among those with potential for full clinical development for treatment of atopic dermatitis. Another humanized antibody suitable for treatment of atopic dermatitis is E25 (rhuMAb-E25), produced by Genentech, Inc. This antibody is described in Presta et al., J. Immunol. 151:2623-2632 (1993).

The foregoing description, terms, expressions and examples are exemplary only and not limiting. The invention includes all equivalents of the foregoing embodiments, both known and unknown. The invention is defined by the claims that follow and is not limited by any statement in any other portion of this document or in any other source.

1. A method for treating or preventing hypersensitivity and/or anaphylaxis in a mammal administered one or more replacement therapy molecules that elicit an IgE-mediated response comprising administering an anti-IgE antibody or a binding fragment thereof to the mammal in an amount sufficient to treat or prevent hypersensitivity and/or anaphylaxis.

2. A method of reducing or preventing an IgE-mediated response in a mammal administered a replacement therapy molecule comprising administering an anti-IgE antibody or a binding fragment thereof to the mammal in an amount sufficient to reduce or prevent the IgE-mediated response.

3. A method of reducing the risk of anaphylaxis in a mammal administered a replacement therapy molecule that elicits an IgE-mediated response comprising administering an anti-IgE antibody or a binding fragment thereof to the mammal in an amount sufficient to reduce the risk of anaphylaxis.

4. A method for inducing immune tolerance in a mammal administered a replacement therapy molecule that elicits an IgE-mediated response comprising administering an effective desensitizing amount of the replacement therapy antigen in combination with an anti-IgE antibody or a binding fragment thereof to the mammal sufficient to induce tolerance to the replacement molecule.

5. A method for inhibiting IgE antibody production in a mammal administered a replacement therapy molecule, which comprises administrating to the mammal an amount of an anti-IgE antibody or a binding fragment thereof sufficient to inhibit IgE antibody production.

6. A method of reducing the amount of replacement therapy molecule needed to effectively treat a disease comprising administering to a mammal an anti-IgE antibody or a binding fragment thereof sufficient to reduce neutralizing IgE thus reducing the amount of replacement molecule needed.

7. A method of administering replacement therapy to a mammal comprising administering a replacement therapy molecule to a mammal, and administering an anti-IgE antibody or a binding fragment thereof to the mammal.

8. The method according to claim 1, wherein the anti-IgE antibody is a monoclonal antibody.

9. The method according to claim 8, wherein the monoclonal antibody is a human antibody, humanized antibody, a chimeric antibody, or a single-chain antibody.

10. The method of claim 8, wherein the monoclonal antibody is administered as a composition further comprising a physiologically acceptable carrier, diluent, excipient, or stabilizer.

11. The method according to claim 1, wherein the binding fragment is a ScFv, Fv, Fab, F(ab′) or F(ab′)₂.

12. The method according to claim 1, wherein the antibody is administered intravenously, intraperitoneally, by inhalation, intramuscularly, subcutaneously or orally.

13. The method according to any one of claims 1 to 4, wherein the IgE-mediated response is an allergic reaction.

14. The method according to claim 1, wherein the replacement therapy molecule is Factor VIII, Factor IX, human alpha-L-iduronidase, human β-glucocerebrosidase or human β-galactosidase A.

15-24. (canceled)

25. A pharmaceutical composition comprising a replacement therapy molecule and an anti-IgE antibody or a binding fragment thereof.
